(19) United States
(12) Patent Application Publication
(10) Pub. No.: US 2015/0132351 A1
(43) Pub. Date: May 14, 2015

(54) USE OF A COMPOSITION CONTAINING A POLYMER AND MINERAL FILLERS TO COMBAT SKIN AGING

(71) Applicant: RHODIA POLIAMIDA E ESPECIALIDADES LTDA, Sao Paolo (BR)

(72) Inventors: Thomas Gonzaga Canova, Sao Paolo (BR); Gabriel Gorescu, Sao Paolo (BR); Tarcis Cordeiro Bastos, Sao Paolo (BR)

(21) Appl. No.: 14/401,986

(22) PCT Filed: May 21, 2013
(86) PCT No.: PCT/IB2013/000992

§ 371 Date: Nov. 18, 2014

(30) Foreign Application Priority Data
May 22, 2012 (FR) 1254662

(51) Int. Cl. A61K 8/88; A61Q 19/08; A61Q 19/02; A61K 8/25; A61Q 1/00; A61K 8/22; A61K 8/24; A61K 8/29; A61K 8/85; A61K 8/81; A61K 8/02; A61K 8/23

(52) U.S. Cl. CPC A61K 8/88; A61K 8/0212; A61K 8/0283; A61Q 19/08; A61Q 19/02; A61K 8/25; A61K 8/23; A61K 8/22; A61K 8/24; A61K 8/29; A61K 8/85; A61K 8/8141; A61Q 1/00; A61K 2800/54; A61K 2800/651
USPC 424/402; 424/709; 424/688; 424/601; 424/617; 424/677; 424/63

(57) ABSTRACT

Methods of using a polymeric composition are described for preventing or reducing the signs of aging. The polymeric composition can include a polymer matrix and one or a plurality of mineral filler(s), uniformly dispersed in the polymer matrix, having properties of absorption and/or emission in the far infrared region ranging from 2 μm to 20 μm.

The subject of the present invention is the use of a polymeric composition containing a polymer matrix and one or more mineral filler(s), uniformly dispersed in this polymer matrix, which have properties of absorption and/or emission of radiation in the far infrared range, in order to prevent or reduce the signs of skin aging.

It is known that the appearance of human skin changes over time, owing to generally natural aging phenomena, which can be accelerated by external factors such as pollution and lifestyle (for example, diet, stress, smoking).

This aging of the skin results in particular in the appearance, at its surface, of various marks such as more or less deep wrinkles, and spots known as “age spots”.

These marks of aging on the skin are increasingly considered to be unattractive, in particular those located on the parts of the body that show, such as the face, the neck or the hands.

Thus, numerous cosmetic products have been developed for preventing the appearance of the signs of skin aging. These products are generally in the form of compositions such as creams, more or less thick fluids, or sera, containing one or more active ingredients which are chemical or natural compounds intended to combat wrinkles and/or spots. These compositions must generally be applied to the areas to be treated once or twice a day, and have very variable degrees of effectiveness.

Despite the numerous products currently present on the market, the consumer is still searching for innovative and effective solutions which can be used in place of or as a supplement to the already existing solutions. There is thus a need to provide new solutions, which make it possible to effectively combat skin aging.

Pursuing its research in this field, the applicant has now discovered a novel and original approach, which makes it possible to effectively combat the signs of skin aging, in particular wrinkles and age spots.

This approach is based on the use of a particular polymeric composition, comprising a polymer matrix within which are dispersed mineral fillers which emit and/or absorb infrared radiation in the wavelength range located between 2 μm and 20 μm.

Indeed, the applicant has discovered, totally unexpectedly, that such a polymeric composition, when it is brought into contact with the skin, has the effect of reducing the signs of aging already present on the skin, and of preventing or delaying the appearance of new signs.

Thus, the subject of the present invention is the use, for preventing or reducing the signs of skin aging, of a polymeric composition containing a polymer matrix and one or more mineral filler(s), uniformly dispersed in the polymer matrix, having properties of absorption and/or emission in the far infrared region ranging from 2 μm to 20 μm.

In a manner known per se, the term “signs of skin aging” denotes the marks present on the skin resulting from aging phenomena, which modify its visual appearance and are generally considered to be unattractive, such as, in particular, wrinkles and age spots.

The subject of the present invention is also a method for preventing or reducing the signs of skin aging, consisting in bringing the skin into contact with a polymeric composition as described in the present application.

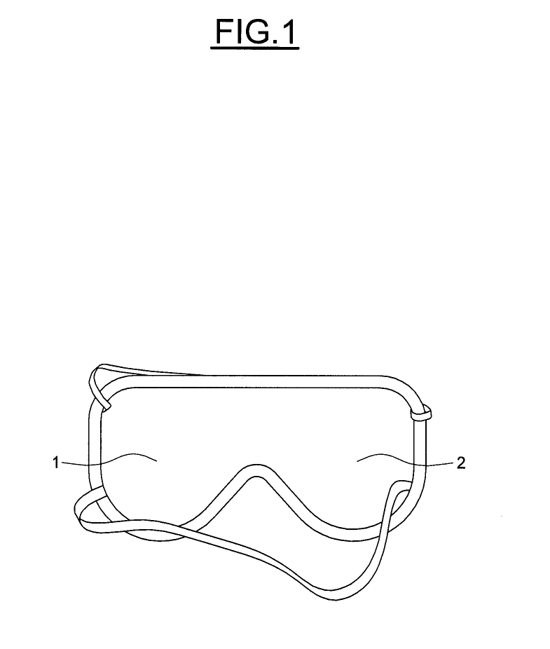

FIG. 1 appended hereto illustrates a mask intended to be applied to the eyes, and which comprises a polymeric composition according to the invention.

The invention uses a polymeric composition comprising a polymer matrix.

The polymer matrix can be chosen in particular from the group comprising: polyesters, polyolefins, polymers based on a cellulose ester, such as cellulose acetate, cellulose propionate, rayon, viscose and polymers of the same family, acrylic polymers and copolymers, polyamides such as polyhexamethylene adipamide (PA66), polycaproamide (PA6), PA6.10, PA10.10 and PA12, copolymers in any proportions of these polymers, and blends between any of these polymers.

According to one preferential embodiment, the polymer matrix consists of polyamide, preferably chosen from polyamide 6, polyamide 66 and copolymers of polyamide 6/polyamide 66 in any proportions.

The composition according to the invention comprises one or more mineral filler(s) having properties of absorption and/or emission in the far infrared region ranging from 2 to 20 μm. Preferably, the mineral filler(s) has (have) properties of absorption and/or emission in the far infrared region ranging from 3 to 20 μm, and even more preferentially from 3 to 15 μm.

According to the invention, the mineral filler(s) is (are) uniformly dispersed in the polymer matrix. The term “uniformly dispersed” is intended to mean that the mineral fillers are homogeneously incorporated actually into the polymer. In particular, the particles are trapped in the polymer composition. They are not therefore mineral fillers deposited on the polymer, for example in the form of a coating at the surface of the polymer.

Such a uniform dispersion can be obtained by incorporating the mineral filler(s) into the polymer during the synthesis of the latter. One embodiment consists in producing one or more surfactant-stabilized suspensions of mineral fillers. The suspension(s) is (are) then added during the synthesis of the polymer.

Said fillers can also be incorporated by mixing the latter with the molten polymer, either directly, or by means of a concentrate of particles in the form of a masterbatch, it being possible for the latter to be subsequently diluted to predetermined concentrations in the polymer mass. This incorporation into the molten polymer can advantageously be carried out at the time of the forming of the polymeric composition, for example at the time of the extrusion of the polymeric composition.

By virtue of such processes, it is possible to obtain polymer compositions according to the invention which contain the mineral filler(s) in a manner uniformly dispersed in the polymer matrix.

The mineral filler(s) usable according to the invention can be chosen in particular from oxides, sulfates, carbonates, phosphates and silicates.

Preferably, the oxide(s) is (are) chosen from titanium dioxide, silicon dioxide and magnesium oxide.

The sulfate(s) can advantageously be chosen from alkali metal and alkaline-earth metal sulfates, preferably from barium sulfate, calcium sulfate and strontium sulfate.

The carbonate(s) is (are) advantageously chosen from calcium carbonate or sodium carbonate.

Preferably, the silicate(s) is (are) chosen from actinolite, tourmaline, serpentine, kaolinite, and zirconium silicate.

The phosphate(s) can be chosen from zirconium phosphates, cerium phosphate and apatite, and mixtures thereof.

Preferably, the polymeric composition contains at least two mineral fillers of different types, chosen from the following types: oxides, sulfates, carbonates, phosphates and silicates. Particularly preferably, the polymeric composition contains at least three mineral fillers of different types, chosen from the abovementioned types.

According to a first preferred embodiment, the polymeric composition contains at least two mineral fillers of different types, chosen from the following types: oxides, sulfates and silicates, and preferably from titanium dioxide, an alkali metal or alkaline-earth metal sulfate and a silicate, and even more preferably from titanium dioxide, barium sulfate and tourmaline.

More preferably, the polymeric composition contains at least three mineral fillers of different types, chosen from the above types. Particularly preferably, the polymeric composition contains three mineral fillers of different types, which are an oxide, a sulfate and a silicate.

Preference is given quite particularly to the titanium dioxide/alkaline-earth metal sulfate/silicate combination, and even more preferentially the titanium dioxide/barium sulfate/tourmaline combination.

In this case, the respective weight proportions of the three mineral fillers above are preferably between 80:10:10 and 10:30:60, and more specifically these respective proportions are 50:25:25.

According to a second embodiment, which is likewise advantageous, the polymeric composition contains at least two mineral fillers of different types, and preferably at least three mineral fillers of different types, chosen from the following types: oxides, phosphates and silicates.

In this embodiment, combinations of three mineral fillers of different types, namely an oxide, a phosphate and a silicate, are particularly preferred.

Preferably, the weight proportion of mineral filler(s) relative to the total weight of the polymeric composition is greater than or equal to 1.0%, preferably greater than or equal to 1.5% and even more preferentially greater than or equal to 2.5%.

Preferably, the weight proportion of mineral filler(s) relative to the total weight of the polymeric composition is less than or equal to 50%, preferably less than or equal to 40% and even more preferentially less than or equal to 30%.

The mineral filler(s) according to the invention is (are) advantageously in the form of particles, which preferably have a volume-average size of less than or equal to 2 μm, measured according to the laser diffraction particle size analysis method (using, for example, Malvern or Cilas particle size analyzers).

One advantageous way to carry out the process consists in suspending the particles in water and in determining their particle size by laser diffraction using the method described in standard ISO 13320:2009.

It is preferable for the mineral fillers used in the present invention to have a particle size which is:
neither too small, so as to prevent any risk of the particles being able to leave the polymer matrix and introduce themselves into the human body through the skin or via the airways, or else disperse in the environment;
nor too large, which would make the incorporation of the particles into the polymer matrix more difficult and especially might make the composition abrasive on contact with the skin, which may be uncomfortable for the user, or even, in certain cases, might cause the risk of an irritant effect on the skin, for example in the case of particularly thin or sensitive skin.

Thus, the mineral filler(s) according to the invention is (are) in the form of particles which advantageously have a volume-average size, measured according to the laser diffraction particle size analysis method, ranging from 0.1 to 2 μm, more preferentially from 0.2 to 1.5 μm and even more preferentially from 0.2 to 1 μm.

The mineral fillers advantageously have a particle size distribution with 99% by volume of the particles having a size of less than 1.0 μm, preferably 90% by volume of the particles having a size of less than 0.5 μm. The particle size distribution is also measured by the abovementioned laser diffraction particle size analysis method (using, for example, Malvern or Cilas particle size analyzers).

The polymeric composition according to the invention preferably has more than 10 infrared radiation absorption peaks in the following 10 frequency ranges: 3.00+/−0.30 μm, 6.20+/−0.50 μm, 8.00+/−0.25 μm, 8.50+/−0.25 μm, 9.00+/−0.25 μm, 9.50+/−0.25 μm, 10.00+/−0.25 μm, 10.50+/−0.25 μm, 11.00+/−0.25 μm, 14.60+/−2.10 μm, at least one peak being present in at least seven of these 10 frequency ranges.

The infrared radiation absorption spectrum can be determined by any method known to those skilled in the art. One possible method is the use of a Bruker Equinox 55 instrument, with a resolution of 4 cm⁻¹. In this case, the spectrum obtained is in ATR (“Attenuated Total Reflectance”) form, using a ZnSe crystal.

The polymeric composition according to the invention can be used in various forms.

Two preferred embodiments of the invention, in which said polymeric composition is used in two preferred types of forms, will be described hereinafter, it being understood that the scope of the invention could not be limited to these two embodiments.

According to a first preferred embodiment of the invention, the polymeric composition is in the form of yarns, fibers or filaments, or in the form of a film.

The yarns, fibers or filaments are preferably incorporated into a textile article, which either consists exclusively of yarns, fibers or filaments consisting of the polymeric composition according to the invention, or comprises such yarns, fibers or filaments, in combination with yarns, fibers or filaments other than those of the invention.

The term “textile article” is intended to mean in particular a fabric, a knit or a nonwoven.

Such a textile article can be produced by known techniques using yarns, fibers or filaments consisting of the polymeric composition according to the invention as starting material, alone or in combination with other yarns, fibers or filaments which may be natural (for example cotton) or synthetic (for example viscose).

In this first embodiment, the textile articles and the films based on the polymeric composition of the invention can have any appropriate shape which makes it possible to bring them into contact with the skin, and to hold them in place on said skin for a sufficiently long period of time. Particularly advantageously, they can have the shape of a mask covering all or part of the face, or of one or more patch(es) to be applied to the areas to be treated.

It is possible, for example, to use a mask which has the shape of that shown in FIG. 1, and which makes it possible to cover the eyelids and the area around the eyes.

In the case where the polymeric composition is in the form of yarns, fibers or filaments, the ratio between the size of particles of the mineral filler(s) and the diameter of the filaments can advantageously be optimized so as to avoid any risk of the particles being too small and being able to leave the polymer matrix and introduce themselves into the human body or disperse in the environment, or, on the contrary, being too large, with the risk of making the composition abrasive on contact with the skin.

Thus, the ratio between the equivalent average diameter of the filaments according to the invention and the volume-average size of the mineral fillers, measured according to the abovementioned laser diffraction particle size analysis method, is then advantageously greater than or equal to 10. This ratio between the equivalent average diameter of the filament and the volume-average size of the mineral fillers is preferably less than or equal to 200.

The yarns, fibers or filaments according to the invention are characterized in that the filaments preferably have a linear mass (or titer) ranging from 0.2 to 20 dtex, advantageously from 0.5 to 8 dtex and even more preferentially from 0.5 to 3.5 dtex.

The filaments according to the invention preferably have an equivalent average diameter ranging from 4 to 50 μm, preferably from 4 to 30 μm and more preferentially from 4 to 20 μm.

The equivalent average diameter of the filaments is advantageously measured by optical microscopy.

The yarns, fibers or filaments according to the invention are prepared using methods well known to those skilled in the art, by spinning the polymeric composition. It is thus possible to obtain continuous multifilament yarns, monofilaments, short or long fibers, or mixtures thereof. Such yarns, fibers and filaments can be subjected to all the textile treatments known to those skilled in the art, such as extrusion, drawing, texturing, dyeing, finishing, etc.

The films according to the invention can be prepared by conventional processes, for example by blow molding or casting in the form of a thin sheet of the polymeric composition. It is possible to use conventional extrusion devices, and to carry out any appropriate post-treatments (for example wetting, annealing).

The yarns, fibers, filaments or films above can advantageously have additional functionalities, different than the functionality of emission/absorption in the far infrared (FIR), for example and in a nonlimiting manner, one or more of the functionalities below:
regulation of moisture content, in particular moisturization of the skin,
protection against microbes,
hydrophobicity/hydrophilicity,
water absorption capacity/capillarity,
anti-odor,
antifungal,
insect repellent,
protection against UV radiation.

These additional functionalities can be provided by additives/active agents, added to the yarns, fibers, filaments or films during the preparation thereof.

According to one particularly advantageous embodiment, it is possible to add, to the yarns, fibers, filaments or films, one or more antiwrinkle active ingredients different than the mineral fillers according to the invention.

As set out above, the textile articles according to the invention can be obtained from a single type of yarn, fiber or filament according to the invention, or from a mixture of yarns, fibers or filaments according to the invention with yarns, fibers or filaments other than those of the invention. The yarns, fibers or filaments other than those of the invention can advantageously have functionalities which are different than and/or additional to the functionality of emission/absorption in the far infrared (FIR). They may in particular be yarns, fibers or filaments with one or more of the functionalities below:
regulation of moisture content, in particular moisturization of the skin,
antimicrobial protection,
hydrophobicity or hydrophilicity,
water absorption capacity/capillarity,
anti-odor,
antifungal,
insect repellent,
protection against UV radiation,
nonstick.

These functionalities can be provided by additives/active agents, added to the yarns, fibers or filaments other than those of the invention during the preparation thereof.

The textile articles and films according to the invention can absolutely be washed, for example with water to which a conventional detergent has optionally been added, and exhibit excellent resistance to washing, with good persistence of their cosmetic properties. In particular, by virtue of the incorporation of the fillers into the polymer matrix, said fillers are not eliminated during washing.

In said first embodiment of the invention, the yarns, fibers or filaments (in particular in the form of a textile article) or the film are used by applying them to the areas of the skin to be treated, then by leaving them in contact with said skin for a sufficient period of time, typically of at least one hour, for example for a period of time ranging from 1 to 12 hours.

For example, when the polymeric composition according to the invention is used in the form of a face mask, it can advantageously be worn at night.

According to one particularly advantageous embodiment, the textile article or the film is used in combination with a conventional cosmetic product, in particular with a cosmetic composition containing one or more antiwrinkle or anti-spot active ingredients, such as, for example, a cream, a serum or a lotion. In this case, the cosmetic composition is advantageously applied to the skin, before placing thereon the textile article or the film according to the invention.

In this embodiment, the applicant has in fact unexpectedly noted a synergistic effect resulting from the use of the polymeric composition according to the invention with the cosmetic compositions containing antiwrinkle or anti-spot active ingredients.

According to a second preferred embodiment of the invention, the polymeric composition is in the form of particles, which can in particular be dispersed in a cosmetic composition such as a cream, a fluid, a serum, or a makeup composition which may be solid (powder, lipstick) or fluid.

In this embodiment, the particles of polymeric composition advantageously have a volume-average size of less than or equal to 250 μm, preferably ranging from 5 to 150 μm and more preferentially from 10 to 50 μm.

The volume-average size of the particles of polymeric composition is measured according to the laser diffraction particle size analysis method described above (using, for example, Malvern or Cilas particle size analyzers).

In this second embodiment, the ratio between the volume-average size of the particles of polymeric composition and the volume-average size of the mineral fillers can also be optimized so as to avoid any risk of the particles being too small and being able to leave the polymer matrix and introduce themselves into the human body or disperse in the environment, or, on the contrary, being too large, with the risk of making the composition abrasive on contact with the skin.

Thus, the ratio between the volume-average size of the particles of polymeric composition according to the invention and the volume-average size of the mineral fillers, these two sizes being measured according to the laser diffraction particle size analysis method, is then advantageously greater than or equal to 5. This ratio is preferably less than or equal to 250. This ratio preferably ranges from 5 to 150 and more preferentially from 5 to 100.

The particles of polymeric composition according to the invention can be prepared by the methods known to those skilled in the art for obtaining powders or fine particles of polymers, for example by milling, cryomilling or spray-drying of the polymeric composition. Alternatively, the method described in patent application FR 2 899 591 in the name of the applicant, the content of which is incorporated into the present application by way of reference, can be used.

As explained above, the particles of polymeric composition according to the invention are advantageously used in the form of a dispersion in a cosmetic composition.

The latter may in particular comprise a solvent which can be chosen from water, organic fluids, and mixtures of water and organic fluids which are miscible or immiscible with water.

The cosmetic composition may also comprise any of the conventional ingredients, known to those skilled in the art as being part of the composition of cosmetic skin creams or fluids, such as, for example, and in a nonlimiting way, thickeners, moisturizing agents, UV-screening agents or antioxidants.

According to one particularly advantageous embodiment, said cosmetic composition also comprises one or more antiwrinkle or anti-spot active agents different than the mineral fillers according to the invention.

Indeed, in this case, a synergistic effect between the particles of polymeric composition according to the invention and the antiwrinkle or anti-spot active ingredients has also been noted.

In this second embodiment of the invention, the cosmetic composition containing the particles of polymeric composition according to the invention is used by applying said composition to the skin, on the area(s) to be treated. This application can be daily, twice daily (for example, morning and evening), or more episodic (every other day, once a week, etc).

After application to the skin, the composition can either be left on, or rinsed off after a leave-on time which can range from a few minutes to a few hours.

The subject of the present invention is also a cosmetic treatment method for the skin, for preventing or reducing the signs of skin aging, consisting in bringing the skin into contact with a polymeric composition as described in the present application.

The detailed description, given above, of the use according to the invention also applies to the method according to the invention. In particular, the method can be advantageously carried out according to the two preferred embodiments described above (on the one hand, yarns, fibers, filaments or films, or, on the other hand, particles).

Exemplary embodiments of the invention are given hereinafter. These examples are given by way of illustration and could not in any way be limiting in nature.

### EXAMPLES

### Example 1

### Production of the Polymeric Composition

A masterbatch of polyamide 66 was prepared by incorporating 20% by weight of infrared-emitting mineral fillers in powder form into polyamide 66 with a relative viscosity (RV) of 43, measured in a solution of formic acid at 90% in water.

The resulting masterbatch is extruded, cooled and granulated.

The resulting granules are remelted and then introduced, during the spinning, into molten polyamide 66 with a relative viscosity (RV) of 43, measured in a solution of formic acid at 90% in water, in a proportion which makes it possible to obtain the desired amount of mineral fillers in the polymer matrix.

Spinning of the Polymer and Production of Masks:

The molten polymeric composition obtained is spun at a temperature between 280° C. and 300° C. (measured in the die), cooled in air (20° C., relative humidity of 65%) and wound at a speed of 4200 m/min so as to obtain a continuous multifilament yarn. The multifilament yarn made up of 68 filaments with a circular cross-section was subsequently textured. The titer of the filament in the final product is 1.2 dtex.

In this way, a yarn of polyamide 66 containing 1.5% by weight of TiO₂ having a volume-average particle size of 0.3 μm, 0.5% by weight of BaSO₄ having a volume-average particle size of 0.25 μm and 0.2% by weight of tourmaline having a volume-average particle size of 0.3 μm, was produced.

The resulting first yarn is subsequently converted into knits using a circular knitting machine.

Likewise, a multifilament yarn was also produced from a polyamide 66 (with a relative viscosity (RV) of 43, measured in a solution of formic acid at 90% in water), containing 1.5% by weight of TiO₂ having a volume-average particle size of 0.3 μm.

The resulting second yarn is also made up of 68 filaments with a circular cross-section and was subsequently textured. The titer of the filament in the final product is 1.2 dtex.

The resulting yarn is also converted into knits using a circular knitting machine.

Masks were subsequently prepared from said knits. These masks are shown in FIG. 1 appended hereto: the left-hand part thereof (1) consists of a knit obtained from the first yarns above, and the right-hand part thereof (2) consists of a knit obtained from the second yarns above.

Mask Test and Results:

The study was carried out on two groups each consisting of 17 female volunteers, meeting all the following criteria: 35 to 55 years old, phototype II to IV according to the Fitzpatrick classification, exhibiting wrinkles on the face.

The first group of participants wore a mask as described above, overnight, for 30 days.

The second group of participants wore a mask as described above, overnight, for 30 days, in combination with an antiwrinkle cream on the market sold under the brand Olay®. The cream was applied by the participants to the area around both eyes, every evening immediately before putting on the mask.

The efficacy of the treatment in terms of reduction of wrinkles was evaluated by image analysis. Digital photographs of the area of skin located at the corner of each eye were taken for each participant, before the beginning of the test, and after the 30 days of the test. These photographs were taken using a Nikon D7000 digital photographic apparatus, equipped with a 60 mm F2.8 AF-D Micro lens, with an exposure time of 1/10 s and an F18 aperture.

The photographs were taken under identical conditions, and of identical areas of skin, so as to be able to perform a comparative analysis of the digital images obtained, before and after the 30 days of wearing the mask. The image analysis was performed by means of the Scion Image software.

The results obtained are shown in the table below:

| | Percentage reduction in wrinkles | |
|---|---|---|
| | Right-hand part | Left-hand part |
| | (PA 66 + | (PA 66 + |
| | TiO₂ alone) | TiO₂/BaSO₄/tourmaline) |
| Group 1 (mask alone) | 1.2% | 2.3% |
| Group 2 (mask + | 6% | 10.1% |
| antiwrinkle cream) | | |

These results show that the use of the mask according to the invention makes it possible to measurably reduce wrinkles right from wearing the mask for 30 days.

This effect is synergistically increased when the mask is used together with a cosmetic antiwrinkle cream.

### Example 2

This example illustrates two additional polymeric compositions, which can be prepared using the same preparation process as that described in example 1:

Composition 2a: polyamide 66 containing the following mineral fillers:
1.5% by weight of TiO₂ (volume-average particle size of 0.3 μm),
0.5% by weight of BaSO₄ (volume-average particle size of 0.25 μm), and
0.5% by weight of tourmaline (volume-average particle size of 0.3 μm).
   Composition 2b: polyamide 66 containing the following mineral fillers:
0.3% by weight of TiO₂ (volume-average particle size of 0.3 μm),
0.6% by weight of BaSO₄ (volume-average particle size of 0.25 μm), and
0.8% by weight of tourmaline (volume-average particle size of 0.3 μm).

These compositions can be used to effectively combat wrinkles, for example in the form of textile articles prepared in the manner described in example 1.

### Example 3

In this example, the starting materials used are the following:
PA66 polymer of relative viscosity 2.6;
Tourmaline (volume-average particle size of 0.8 μm);
Barium sulfate (volume-average particle size of 0.8 μm);
Titanium dioxide (volume-average particle size of 0.3 μm);
Additive A: Polyamide/polyalkylene oxide hydrophilic star copolymer obtained in the following way:

The following are introduced into a 7.5-liter autoclave equipped with a mechanical stirrer: 1116.0 g of ε-caprolactam (9.86 mol), 57.6 g of 1,3,5-benzenetricarboxylic acid (0.27 mol), 1826.4 g of Jeffamine M2070 (0.82 mol), 1.9 g of Ultranox 236 and 3.5 g of an aqueous 50% (w/w) solution of hypophosphorous acid.

The reaction mixture is brought to 250° C. under nitrogen and at atmospheric pressure and maintained at this temperature for 1 h. The system is then gradually placed under vacuum for 30 min until a pressure of 5 mbar is obtained, and then maintained under vacuum for a further one hour. The system is then poured onto a plate.
Polyethylene oxide having a molecular weight of 400 g/mol.

### Preparation of the Polymer Composition:

The polyamide is mixed with the tourmaline, the barium sulfate and the titanium dioxide in such a way that the final weight composition is 70% of PA66, 2.7% of tourmaline, 6.8% of barium sulfate and 20.5% of titanium dioxide. The mixture is remelted in a twin-screw extruder at a temperature of 290° C. and extruded so as to obtain the granulated polymer.

### Preparation of Particles of Polymeric Composition 10 μm in Size:

The following are introduced into a 24D twin-screw extruder of Prism type: granules of the polymer composition obtained above, using feeding by volume, and a mixture of pellets of the additive A (weight concentration of 5%) and of polyethylene oxide (weight concentration of 19%), using feeding by weight. The mixture is extruded at a fixed flow rate of 2.0 kg/hour. The temperatures of the various zones of the extruder are between 275 and 295° C. The speed is set at 200 rpm. The pressure recorded is between 10 and 13 bar. The rods obtained are quenched at the die outlet with a stream of water, collected in a metal basket, drained and then dried.

The rods collected are then dispersed in water by simple mechanical stirring. The resulting dispersion is sieved with a 200 μm sieve to remove the large solid impurities, such as nondispersible pieces of rod. The yields by weight for recovery of polyamide polymer after sieving are greater than 90%. The particle size distribution of the particles contained in the dispersion was measured using a MasterSizer 2000 device sold by the company Malvern Instruments. This distribution, expressed by volume, obtained after application of ultrasound, is unimodal and the value of the modal peak is 10 μm.

Particles of polymeric composition containing 70% by weight of PA66 and 30% by weight of mineral fillers (titanium dioxide, barium sulfate, tourmaline) are thus obtained.

### Preparation of an Antiwrinkle Cosmetic Composition Containing the Particles of Polymeric Composition:

An antiwrinkle face composition was prepared from the ingredients indicated in the table below (the content of each ingredient being indicated as a percentage by weight, relative to the total weight of the composition).

The particles of polymeric composition used are those prepared according to the description above.

| | Content |
|---|---|
| Ingredients | by weight |
| Phase A | |
| Cyclopentasiloxane/PEG/PPG-20/15 Dimethicone | 11.00% |
| (product sold under the name SF1528 by the company | |
| Kobo Products) | |
| Cyclopentasiloxane (product sold under the name SF1202 | 9.00% |
| by the company Kobo Products) | |
| Cyclopentasiloxane/Dimethicone (product sold under the | 7.50% |
| name SF1214 by the company Kobo) | |
| Phase B | |
| Particles of polyamide 66 containing mineral fillers | 7.50% |
| Phase C | |
| Glycerol (Glycerin U.S.P. Natural 96%, sold by the | 8.00% |
| company Univar USA Inc.) | |
| Sodium chloride | 1.00% |
| Butylene glycol/Water/Palmitoyl hydroxypropyltrimonium | 0.50% |
| amylopectin/Glycerol polymer/Polysorbate20/Retinol/ | |
| Phenoxyethanol/Parabens/Hydrogenated | |
| lecithin/BHT/BHA (product sold under the name Gs- | |
| VA100C by the company Kobo Products) | |
| Water/Papain/Palmitoyl hydroxypropyltrimonium | 0.50% |
| amylopectin/Crosslinked glycerol polymer/ | |
| Phenoxyethanol/Hydrogenated lecithin/Parabens | |
| (product sold under the name GsPPY by the company | |
| Kobo Products) | |
| Polysorbate 80 (product sold under the name Liposorb O- | 0.20% |
| 20 by the company LIPO Chemicals) | |
| Quaternium-15 (product sold under the name Dowicil 200 | 0.10% |
| by the company DOW Chemical) | |
| Deionized water | qs 100% |

This composition was prepared in the following way: the compounds of phase A were mixed, and the mixture was homogenized for 15 minutes. The polymer particles (phase B) were then added, and the homogenization was continued for 15 minutes.

The ingredients of phase C were premixed separately, then gradually added to the main mixture in five portions, while observing a mixing time of 15-20 minutes between each addition.

After complete homogenization of the mixture, the resulting composition was then packaged, by pouring it into appropriate containers.

1. A method of reducing signs of aging, the method comprising administering to an individual subject in need thereof a polymeric composition comprising a polymer matrix and one or more mineral filler(s), uniformly dispersed in the polymer matrix, having properties of absorption and/or emission in the far infrared region ranging from 2 μm to 20 μm.

2. The method as claimed in claim 1, wherein the method reduces wrinkles and/or age spots.

3. The method as claimed in claim 1, wherein the polymer matrix is selected from the group consisting of polyesters, polyolefins, polymers based on cellulose esters, acrylic polymers and copolymers, polyamides, copolymers thereof and blends thereof.

4. The method as claimed in claim 1, wherein the polymer matrix comprises a polyamide.

5. The method as claimed in claim 1, wherein the polymeric composition comprises at least two mineral fillers of different types selected from the group consisting of: oxides, sulfates, carbonates, phosphates and silicates.

6. The method as claimed in claim 1, wherein the polymeric composition comprises at least two mineral fillers of different types selected from the group consisting of: oxides, sulfates and silicates.

7. The method as claimed in claim 1, wherein the polymeric composition comprises three mineral fillers of different types, which are an oxide, a sulfate and a silicate.

8. The method as claimed in claim 5, wherein the polymeric composition comprises at least two mineral fillers of different types selected from the group consisting of: oxides, phosphates and silicates.

9. The method as claimed in claim 1, wherein the polymeric composition comprises three mineral fillers of different types, which are an oxide, a phosphate and a silicate.

10. The method as claimed in claim 1, wherein the weight proportion of mineral filler(s) relative to the total weight of the polymeric composition is greater than or equal to 1.0%.

11. The method as claimed in claim 1, wherein the weight proportion of mineral filler(s) relative to the total weight of the polymeric composition is less than or equal to 50%.

12. The method as claimed in claim 1, wherein the mineral filler(s) is (are) in the form of particles which have a volume-average size, measured according to the laser diffraction particle size analysis method, of less than or equal to 2 μm.

13. The method as claimed in claim 1, wherein the polymeric composition is in the form of yarns, fibers or filaments.

14. The method as claimed in claim 32, wherein the textile article or the film has the shape of a mask or a patch.

15. The method as claimed in claim 1, wherein the polymeric composition is in the form of particles, dispersed in a cosmetic composition.

16. The method as claimed in claim 15, wherein the particles of the polymeric composition have a volume-average size, measured according to the laser diffraction particle size analysis method, of less than or equal to 250 μm.

17. (canceled)

18. The method as claimed in claim 4, wherein the polyamide is selected from the group consisting of polyamide 6, polyamide 66 and copolymers of polyamide 6/polyamide 66 in any proportions.

19. The method as claimed in claim 5, wherein the polymeric composition comprises at least three mineral fillers of the different types.

20. The method as claimed in claim 6, wherein the polymeric composition comprises at least three mineral fillers of different types.

21. The method as claimed in claim 6, wherein different types of fillers are selected from the group consisting of titanium dioxide, an alkali metal or alkaine-earth metal sulfate and a silicate.

22. The method as claimed in claim 6, wherein the different types of fillers are selected from the group consisting of titanium dioxide, barium sulfate and tourmaline.

23. The method as claimed in claim 7, wherein the three mineral fillers of different types are a titanium dioxide/barium sulfate/tourmaline combination.

24. The method as claimed in claim 8, wherein the polymeric composition comprises at least three mineral fillers of different types.

25. The method as claimed in claim 10, wherein the weight proportion of the mineral filler(s) is greater than or equal to 1.5%.

26. The method as claimed in claim 10, wherein the weight proportion of the mineral filler(s) is greater than or equal to 2.5%.

27. The method as claimed in claim 11, wherein the weight proportion of the mineral filler(s) is less than or equal to 40%.

28. The method as claimed in claim 11, wherein the weight proportion of the mineral filler(s) is less than or equal to 30%.

29. The method as claimed in claim 12, wherein the volume-average size of the particles ranges from 0.1 μm to 2 μm.

30. The method as claimed in claim 12, wherein the volume-average size of the particles ranges from 0.2 μm to 1.5 μm.

31. The method as claimed in claim 12, wherein the volume-average size of the particles ranges from 0.2 μm to 1 μm.

32. The method as claimed in claim 13, wherein the yarns, fibers or filaments are incorporated into a textile article or are in the form of a film.

33. The method as claimed in claim 15, wherein the cosmetic composition is a cream, a fluid, a serum or a solid or fluid makeup composition.

34. The method as claimed in claim 16, wherein the volume-average size of the particles ranges from 5 μm to 150 μm.

35. The method as claimed in claim 16, wherein the volume-average size of the particles ranges from 10 μm to 50 μm.
